(19) United States
(12) Patent Application Publication
(10) Pub. No.: US 2006/0167068 A1
(43) Pub. Date: Jul. 27, 2006

(54) Method of treating self-injurious behavior with glutamate modulating agents

(76) Inventors: Seth Feuerstein, Woodbridge, CT (US); Vladimir Coric, Madison, CT (US)

Correspondence Address: CANTOR COLBURN, LLP, 55 GRIFFIN ROAD SOUTH, BLOOMFIELD, CT, 06002, US

(21) Appl. No.: 11/339,881

(22) Filed: Jan. 26, 2006

Related U.S. Application Data
(60) Provisional application No. 60/647,535, filed on Jan. 26, 2005.

(51) Int. Cl. A61K 31/428; A61K 31/198

(52) U.S. Cl. 514/367; 514/562

(57) ABSTRACT

Inventors have made the surprising discovery that glutamate modulating agents are useful for treating borderline personality disorder and self-injurious behavior. Methods of treating borderline personality and self-injurious behavior are provided herein by administering a glutamate modulating agent to a patient are included herein. The invention also includes combination methods of treatment in which a glutamate modulating agent is administered with one or more other CNS active agent. Packaged pharmaceutical compositions containing a glutamate modulating agent and one or more other CNS agent are provided by the invention as are and packaged pharmaceutical formulations containing a glutamate modulating agent and instructions for using the glutamate modulating agent for treating borderline personality disorder or self-mutilating behavior.

### CROSS REFERENCE TO RELATED APPLICATIONS

This application claims priority from U.S. Provisional Appl. No. 60/647,535 filed Jan. 26, 2005, which is hereby incorporated by reference in its entirety.

### FIELD OF INVENTION

Methods of treating borderline personality and self-injurious behavior by administering a glutamate modulating agent to a patient are included herein. The invention also includes combination methods of treatment in which a glutamate modulating agent is administered with one or more other CNS active agent/s. Packaged pharmaceutical compositions containing a glutamate modulating agent and one or more other CNS agent are provided by the invention as are packaged pharmaceutical formulations containing a glutamate modulating agent and instructions for using the glutamate modulating agent for treating borderline personality disorder or self-mutilating behavior.

### BACKGROUND

Borderline personality disorder is one of ten types of personality disorders listed in the Diagnostic and Statistical Manual of Mental Disorders (DSM-IV). It is characterized by a pattern of instability in intrapersonal relationships and impulsivity. Persons with borderline personality disorder typically exhibit five or more of the following types of impulsivity: frantic efforts to avoid real or imagined abandonment; unstable interpersonal relationships; identity disturbance; self-damaging impulsivity in areas such as sex, spending, substance abuse, binge eating, and reckless driving; recurrent suicide attempts; self-injurious behavior; problems with anger management; hyper-reactivity of mood such as intense anxiety or irritability; and transient paranoid ideation or dissociative symptoms.

Some individuals with this disorder may respond to treatment with anti-depressant, anti-psychotic, or anxiolytic medications.

Self-injurious behavior is behavior in which individuals consciously or unconsciously inflict various types of bodily harm on themselves without intent to commit suicide. Self-injurious behavior is also referred to as self-harm, self-mutilation, and self-injury, and self-inflicted violence. Common types of self-injurious behavior include shallow cuts to the skin on the arms and legs or other parts of the body. Individuals engaging in self-injurious behavior may also hit, bite, or burn themselves. Some type of emotional pressure or the need to relieve physical discomfort may drive the desire to self-injure.

Self-injurious behaviors represent a difficult to treat and often clinically dangerous problem. Moreover, limited pharmacological interventions exist to directly target an individual's self-injurious behaviors or desire to cut. Self-injurious behavior may be associated with borderline personality disorder, but may also be found in patients who do not have borderline personality disorder. Self-injurious behavior has been noted in mentally retarded patients with cortical damage, autistic individuals, individuals with eating disorders, and individuals with seizure disorders, as well as in individuals who present no other psychiatric symptoms.

The neural circuitry underlying the self-injurious behavior and borderline personality disorder is poorly understood. While some animal data suggests that glutamatergic systems are involved in self-injurious behavior, multiple neurotransmitter systems, including the dopaminergic, opioidergic, and serotonergic systems, have been implicated in these disorders. Individuals with these disorders are treated with a variety of CNS active drugs and psychotherapy. However many individuals with borderline personality disorder or self-injurious behavior cannot be adequately treated with current therapies. Therefore there exists a need for improved methods of treating borderline personality disorder and self-injurious behavior.

Riluzole, sold under the trade name RILUTEK, is indicated for treatment of amyotrophic lateral sclerosis and is a potent anti-glutamatergic agent. It is thought to work by reducing synaptic release of glutamate, inactivating voltage-gated sodium channels in cortical neurons, and reducing the excitatory-inhibitory balance in the brain by inhibiting gamma-aminobutyric acid reuptake. Altered glutamatergic neurotransmission has been implicated in the pathophysiology of mood and anxiety disorders. Some studies also suggest that riluzole is efficacious in the treatment of psychiatric disorders such as depression and obsessive compulsive disorder.

N-acetylcysteine is an amino acid often used for its antioxidant properties. It is also thought to modulate glutamate by stimulating the cysteine-glutamate antiporter located on glia, increasing extrasynaptic glutamate levels and thereby stimulating the feedback inhibition of synaptic glutamate release.

The present invention fulfills the need for a new method of treating borderline personality disorder and self-injurious behavior and provides further advantages described herein.

### SUMMARY OF THE INVENTION

The invention pertains to the use of glutamate modulating agents (including riluzole and N-acetyl cysteine), alone or in combination, in the treatment of personality disorders, borderline personality disorder, self-injurious behaviors, cutting, cravings to cut, desire to injure one's self, or addictive behaviors.

The invention includes a method of treating borderline personality disorder or self-injurious behavior in a patient in need thereof by administering an effective amount of a glutamate modulating agent. In some instances the self-injurious behavior is associated with borderline personality disorder. The glutamate modulating agent used in this method may be any agent shown to alter glutamate levels in vivo or in vitro or shown to modulate a cellular response to glutamate. Examples of glutamate modulating agents include, but are not limited to, riluzole and N-acetyl cysteine.

The invention also includes combination methods of treatment in which borderline personality disorder or self-injurious behavior is treated by administering the glutamate modulating agent together with one or more other CNS active agents. In some combination methods of treatment provided by the invention the other CNS active agent is an anti-psychotic, an anti-convulsant, a tricyclic antidepressant, a monoamine oxidase inhibitor, a selective serotonin reuptake inhibitor, a selective serotonin-norepinephrine reuptake inhibitor, a norepinephrine dopamine reuptake inhibitor, a serotonin-2 antagonist reuptake inhibitor, a benzodiazepine, a wakefulness promoting agent, anti-manic agent, or a combination of one or more of the foregoing.

The invention further includes methods of using riluzole and N-acetyl cysteine comprising informing a user that the riluzole or N-acetyl cysteine may be used to treat borderline personality disorder or self-injurious behavior. This method may additionally comprise providing the user with riluzole or N-acetyl cysteine. The user may be informed of the usefulness of riluzole, N-acetyl cysteine, or other glutamate modulating agent for the treatment of borderline personality disorder or self-injurious behavior by reference to a package insert associated with the container. The informing may also be by reference to information material; by reference to a package active agent insert, a flyer or an advertisement; by presentation of information at a seminar, conference, or other educational presentation; or by a conversation between a pharmaceutical sales representative and a medical care worker.

The invention further provides a pharmaceutical composition comprising a glutamate modulating agent such as riluzole or N-acetyl cysteine, and one or more other CNS active agents combined in a single dosage form. Preferably the single dosage form is a dosage form suitable for oral administration such as a tablet, capsule, or syrup. In some embodiments the other pharmaceutical agent used in the pharmaceutical composition is an anti-psychotic, an anti-convulsant, a tricyclic antidepressant, a monoamine oxidase inhibitor, a selective serotonin reuptake inhibitor, a selective serotonin-norepinephrine reuptake inhibitor, a norepinephrine dopamine reuptake inhibitor, a serotonin-2 antagonist reuptake inhibitor, a benzodiazepine, a wakefulness promoting agent, an anti-manic agent or a combination of one or more of the foregoing.

The invention further includes an article of manufacture comprising a glutamate modulating agent in a container and printed labeling stating that the glutamate modulating agent is useful for treating a borderline personality disorder or self-injurious behavior. The glutamate modulating agent present in this article of manufacture may be riluzole, N-acetyl cysteine, or some other glutamate modulating agent. In certain embodiments the printed labeling is labeling approved by the United States FDA.

### DETAILED DESCRIPTION

Terminology

The terms “a” and “an” do not denote a limitation of quantity, but rather denote the presence of at least one of the referenced item.

An “active agent” means any compound, element, or mixture that when administered to a patient alone or in combination with another agent confers, directly or indirectly, a physiological effect on the patient. When the active agent is a compound, salts, solvates (including hydrates) of the free compound or salt, crystalline and non-crystalline forms, as well as various polymorphs of the compound are included. Compounds may contain one or more asymmetric elements such as stereogenic centers, stereogenic axes and the like, e.g. asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers in pure form and mixtures thereof are encompassed. In addition, compounds with carbon-carbon double bonds may occur in Z- and E-forms, with all isomeric forms of the compounds being included in the present invention. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. A “dosage form” means any unit of administration of an active agent.

“Efficacy” means the ability of an active agent administered to a patient to produce a therapeutic benefit in the patient.

“The term glutamate modulating agents” refers to any medication/compound/amino acid/anticonvulsant whose mechanism of action involves the modulation of glutamate levels, glutamate compounds, glutamatergic neurotransmission (i.e. effects the release of glutamate from pre- or post-synaptic neurons, or acts upon glutamate receptors (including NMDA, AMPA, kainate, or metabotropic glutamate receptors)) or excitatory neurotransmission. Glutamate release inhibitors and glutamate modulating agents that that increase clearance of glutamate from the synaptic cleft are particularly effective in treating self-injurious behavior. Both N-Acetyl Cysteine and riluzole are release inhibitors that inhibit presynaptic release of glutamate and also enhance synaptic clearance of glutamate.

“Informing” in any of the above embodiments of the invention may occur by reference to, or providing, information material. Informing can also occur by presentation at a seminar, conference, or other educational presentation; or by providing an active agent with informational material to a user; or in a conversation between a pharmaceutical sales representative and a medical care worker or between a medical care worker and a patient.

“Informational material” means any media providing information. Media includes printed, audio, visual, or electronic media. Examples of information material are flyer, an advertisement, a package insert for a pharmaceutical product, printed labeling, an internet web site, an internet web page, an internet pop-up window, or information recorded on a compact disk, DVD, an audio recording, or any other recording or electronic medium.

A “medical care worker” means any worker in the health care field who may need information regarding an active agent, including information on safety, efficacy, dosing, administration, or pharmacokinetics. Examples of medical workers include physicians, pharmacists, physician's assistants, nurses, caretakers, emergency medical workers, and veterinarians.

A “patient” means any human or non-human animal in need of medical treatment. Medical treatment can include treatment of an existing condition, such as a disease or disorder, prophylactic or preventative treatment, or diagnostic treatment. In some embodiments the patient is a human patient.

As used herein “a pharmaceutical supplier” means any person (other than a medical care worker), business, charitable organization, governmental organization, or other entity involved in the transfer of active agent between entities, for profit or not. Examples of pharmaceutical suppliers include pharmaceutical distributors, pharmacies (online or physical), foreign businesses or individuals importing active agent into the United States, the hospitals, HMOs and the Veterans Administration.

“Pharmaceutically acceptable salts” includes derivatives of the disclosed compounds, wherein the parent compound is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts.

Pharmaceutically acceptable organic salts include salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like, and combinations comprising one or more of the foregoing salts.

“Providing” includes giving, selling, distributing, transferring (for profit or not), manufacturing, compounding or dispensing.

A “product” or “pharmaceutical product” is a dosage form of an active agent plus published material and optionally packing.

“Riluzole” is a chemical compound, also sold by the trade name RILUTEK. It has the chemical formula:

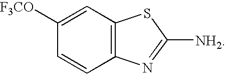

Riluzole is typically administered as a free base but includes all pharmaceutically acceptable salts and hydrates. The term “riluzole” also encompasses all polymorphs and hydrates of this drug.

“Safety” means the incidence of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

The term “therapeutically effective amount” or “effective amount” means an amount effective, when administered to a human or non-human patient, to provide any therapeutic benefit. A therapeutic benefit may be an amelioration of symptoms, e.g., an amount effective to decrease the symptoms of borderline personality disorder or self-injurious behavior. In certain circumstances a patient may not present symptoms of a condition for which the patient is being treated. Thus a therapeutically effective amount of a compound is also an amount sufficient to provide a significant positive effect on any indicia of a disease, disorder or condition e.g. an amount sufficient to significantly reduce the frequency and severity of self-injurious behavior. A significant effect on an indicia of a disorder or condition is typically a statistically significant in a standard parametric test of statistical significance such as Student's T-test, where p≦0.05 though the effect need not be significant in some embodiments.

As used herein, a “user” means a patient, a medical care worker, or a pharmaceutical supplier.

Methods of Treatment

The invention includes a method of treating borderline personality disorder or self-injurious behavior in a patient in need thereof by administering an effective amount of a glutamate modulating agent. In some instances the self-injurious behavior is associated with borderline personality disorder. The glutamate modulating agent used in this method may be any agent shown to alter glutamate levels in vivo or in vitro or shown to modulate a cellular response to glutamate. Examples of glutamate modulating agents include, but are not limited to, riluzole and N-acetyl cysteine.

Frequency of dosage may vary depending on the compound used and the particular condition or disorder to be treated or prevented. In general, for treatment of most personality disorders, including self-injurious behavior, a dosage regimen of 4 times daily or less is preferred; a dosage regimen of 1 or 2 times daily is particularly preferred. In some embodiments employing riluzole in the treatment of a personality disorder, border-line personality disorder, or self-injurious behavior 10 mg to 300 mg riluzole per day or 20 to 200 mg riluzole per day, or about 100 mg per day riluzole is administered. Riluzole is typically administered two times per day in equal doses so each at each administration of riluzole 5 mg to 150 mg riluzole, 10 to 100 mg riluzole, or 50 mg riluzole are given.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease in the patient undergoing therapy. In certain embodiments, administration at meal times is preferred. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

Combination Methods

In a separate aspect, the present invention provides methods of treating a personality disorder, borderline personality disorders, and self-injurious behavior in which the glutamate modulating agent is given in combination with one or more other CNS active agent. Embodiments in which the other CNS active agent(s) are given at the same time as the glutamate modulation agent or given separately are within the scope of the invention. The other CNS active agent may be combined in the same dosage form as the glutamate modulating agent or may be given in a separate dosage form.

The other active agent administered with the glutamate modulating agent is typically a chemical compound known to be efficacious for treating personality disorders. Thus included herein are methods of treating borderline personality disorder or self-injurious behavior, comprising administering the glutamate modulating agent in combination with an antipsychotic, anti-convulsant, tricyclic antidepressant, a monoamine oxidase inhibitor, a selective serotonin reuptake inhibitor, a selective serotonin norepinephrine reuptake inhibitor, a norepinephrine dopamine reuptake inhibitor, a serotonin-2 antagonist reuptake inhibitor, a benzodiazepine, a wakefulness promoting agent, or an anti-manic agent. Some CNS active agents useful treating borderline personality disorder or self-injurious behavior may appear in more than one category. For example clonazepam is both an anti-convulsant and a benzodiazepine.

Anti-psychotics include atypical anti-psychotics. Non-limiting examples of anti-psychotics include clozapine, olanzapine, risperidone, aripiprazole, amisulpride, and loxazpine.

Anti-convulsants include, but are not limited to, anti-epileptics and anti-seizure medications. Non-limiting examples of anti-convulsants include, clonazepam, gabapentin, lamotrigine, and topiramate.

Tricyclic antidepressants include, but are not limited to, doxepin, amitriptyline, amoxapine, clomipramine, desipramine, doxepin, imipramine, maprotiline, nortriptyline, protriptyline, and trimipramine.

Monoamine oxidase inhibitors include, but are not limited to, isocarboxazid, phenelzine, and tranylcypromine.

Serotonin selective reuptake inhibitors, include, but are not limited to, citalopram, clovoxamine, duloxetine, escitalopram, femoxetine, flesinoxan, fluoxetine, fluvoxamine, paroxetine, setraline, trazodone, and zimeldine.

Selective serotonin norepinephrine reuptake inhibitors include, but are not limited to, atomoxetine, mirtazapine, and venlafaxine.

Serotonin-2 antagonist reuptake inhibitors include, but are not limited to, nefazodone

Norepinephrine dopamine reuptake inhibitors include, but are not limited to bupropion.

Benzodiazepines include but are not limited to, alprazolam, clonazepam, diazepam, lorazepam, flurazepam, flumazenil, and bentazepam.

Anti-manics include, but are not limited to, carbamazepine and lithium.

Wakefulness promoting agents include modafinil.

The other active agent may be an NK-1 receptor antagonist, such as, but not limited to aprepitant.

Articles of Manufacture

The invention includes articles of manufacture, which comprise a glutamate modulating agent in a container and labeling stating that the glutamate modulating agent is useful for treating a borderline personality disorder or self-injurious behavior. The glutamate modulating agent may be in the form of a single dosage form; embodiments in which the glutamate modulating agent is in the form of a dosage form suitable for administration are particularly included. The glutamate modulating agent present in this article of manufacture may be riluzole, N-acetyl cysteine, or some other glutamate modulating agent. The article of manufacture may comprise packaging material and a dosage form of a glutamate modulating agent contained within the packaging material, wherein the packaging material comprises a label approved by a regulatory agency for the product. In certain embodiments the labeling is labeling approved by the United States FDA.

An example of an article of manufacture provided by the invention is a packaged pharmaceutical compositions comprising a glutamate modulating agent in a container and printed labeling stating that the glutamate modulating agent is useful for treating a borderline personality disorder or self-injurious behavior. The article of manufacture, for example a packaged pharmaceutical formulation, may optionally comprise one or more other CNS active agent. When the glutamate modulating agent is riluzole, the labeling may advise administering. 10 mg to 300 mg riluzole per day, 20 to 200 mg riluzole per day, or about 100 mg per day. The labeling may advise that riluzole is to be administered one to four times per day.

When the article of manufacture contains one or more other CNS active agent the one or more other CNS active agent and the glutamate modulating agent may be combined in a single dosage form or present in separate dosage forms.

Pharmaceutical compositions in which the glutamate modulating agent is present as a dosage form suitable for oral administration are disclosed herein.

The invention includes providing prescribing information, for example, to a patient or health care provider, or as a label in a packaged pharmaceutical composition. Prescribing information may include for example efficacy, dosage and administration, contraindication and adverse reaction information pertaining to the pharmaceutical composition.

Pharmaceutical Preparations

A glutamate modulating agent alone or in combination with one or more other CNS active agent can be administered as the neat chemical, but are preferably administered as a pharmaceutical composition or formulation. Accordingly, the invention provides pharmaceutical formulations comprising a glutamate modulating agent alone or in combination with one or more other CNS active agent together with one or more pharmaceutically acceptable carrier, excipients, adjuvant, diluent, or other ingredient. Pharmaceutical formulations comprising riluzole have been previously described in U.S. Pat. No. 5,527,814, which is hereby incorporated by reference at cols. 2-3 for its teachings regarding riluzole formulations. Riluzole may be formulated by the methods given in U.S. Pat. No. 5,527,814 for the methods, compositions, and articles of manufacture of this invention or by other means known in the art.

A glutamate modulating agent alone or in combination with one or more other CNS active agent may be administered orally, topically, parenterally, by inhalation or spray, sublingually, transdermally, via buccal administration, or by other means, in dosage unit formulations containing conventional non-toxic pharmaceutically acceptable carriers, excipients, adjuvants, and vehicles. Oral dosages forms such as tablets, troches, lozenges, aqueous or oily suspensions, dispersible powders or granules, emulsions, hard or soft capsules, or syrups or elixirs. In some embodiments solid oral dosage forms are preferred. Compositions intended for oral use may be prepared according to any method known to the art for the manufacture of pharmaceutical compositions and such compositions may contain one or more agents, such as sweetening agents, flavoring agents, coloring agents and preserving agents, in order to provide pharmaceutically elegant and palatable preparations.

Oral formulations contain between 0.1 and 99%, at least about 5% (weight %), 25% to about 50% or from 5% to 75% of a glutamate modulating agent alone or in combination with one or more other CNS active agent and usually at least about 5% (weight %) of a compound of the present invention.

In addition to the glutamate modulating agent alone or in combination with one or more other CNS active agent, the compositions of the invention may contain a pharmaceutically acceptable carrier, one or more compatible solid or liquid filler diluents or encapsulating substances, which are suitable for administration to an animal. Carriers must be of sufficiently high purity and sufficiently low toxicity to render them suitable for administration to the animal being treated. The carrier can be inert or it can possess pharmaceutical benefits of its own. The amount of carrier employed in conjunction with the compound is sufficient to provide a practical quantity of material for administration per unit dose of the compound.

Exemplary pharmaceutically acceptable carriers or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, and corn oil; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

In particular, pharmaceutically acceptable carriers for systemic administration include sugars, starches, cellulose and its derivatives, malt, gelatin, talc, calcium sulfate, vegetable oils, synthetic oils, polyols, alginic acid, phosphate buffer solutions, emulsifiers, isotonic saline, and pyrogen-free water. Preferred carriers for parenteral administration include propylene glycol, ethyl oleate, pyrrolidone, ethanol, and sesame oil.

Optional active agents may be included in a pharmaceutical composition, which do not substantially interfere with the activity of the compound of the present invention.

Effective concentrations of a glutamate modulating agent alone or in combination with one or more other CNS active agent including pharmaceutically acceptable salts, esters or other derivatives thereof are mixed with a suitable pharmaceutical carrier, excipients, adjuvant, or vehicle. In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as Tween, or dissolution in aqueous sodium bicarbonate. Derivatives of the compounds, such as salts of the compounds or prodrugs of the compounds may also be used in formulating effective pharmaceutical compositions.

Upon mixing or addition of the glutamate modulating agent alone or in combination with one or more other CNS active agent, the resulting mixture may be a solution, suspension, emulsion or the like. The form of the resulting mixture depends upon a number of factors, including the intended mode of administration and the solubility of the compound in the chosen carrier or vehicle. The effective concentration sufficient for ameliorating the symptoms of the disease, disorder or condition treated and may be empirically determined.

Liquids Formulations

Compounds useful in the invention can be incorporated into oral liquid preparations such as aqueous or oily suspensions, solutions, emulsions, syrups, or elixirs, for example. Moreover, formulations containing these compounds can be presented as a dry product for constitution with water or other suitable vehicle before use. Such liquid preparations can contain conventional additives, such as suspending agents (e.g., sorbitol syrup, methyl cellulose, glucose/sugar, syrup, gelatin, hydroxyethyl cellulose, carboxymethyl cellulose, aluminum stearate gel, and hydrogenated edible fats), emulsifying agents (e.g., lecithin, sorbitan monsoleate, or acacia), non-aqueous vehicles, which can include edible oils (e.g., almond oil, fractionated coconut oil, silyl esters, propylene glycol and ethyl alcohol), and preservatives (e.g., methyl or propyl p-hydroxybenzoate and sorbic acid).

Orally administered compositions also include liquid solutions, emulsions, suspensions, powders, granules, elixirs, tinctures, syrups, and the like. The pharmaceutically acceptable carriers suitable for preparation of such compositions are well known in the art. Oral formulations may contain preservatives, flavoring agents, sweetening agents, such as sucrose or saccharin, taste-masking agents, and coloring agents.

Typical components of carriers for syrups, elixirs, emulsions and suspensions include ethanol, glycerol, propylene glycol, polyethylene glycol, liquid sucrose, sorbitol and water. Syrups and elixirs may be formulated with sweetening agents, for example glycerol, propylene glycol, sorbitol or sucrose. Such formulations may also contain a demulcent.

Suspensions

Typical suspending agents include methylcellulose, sodium carboxymethyl cellulose, Avicel RC-591, tragacanth and sodium alginate; typical wetting agents include lecithin and polysorbate 80; and typical preservatives include methyl paraben and sodium benzoate.

Aqueous suspensions contain the active material(s) in admixture with excipients suitable for the manufacture of aqueous suspensions. Such excipients are suspending agents, for example sodium carboxymethylcellulose, methylcellulose, hydropropylmethylcellulose, sodium alginate, polyvinylpyrrolidone, gum tragacanth and gum acacia; dispersing or wetting agents; may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol substitute, or condensation products of ethylene oxide with partial esters derived from fatty acids and hexitol anhydrides, for example polyethylene sorbitan substitute. The aqueous suspensions may also contain one or more preservatives, for example ethyl, or n-propyl p-hydroxybenzoate.

Oily suspensions may be formulated by suspending the active ingredients in a vegetable oil, for example peanut oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Emulsions

Pharmaceutical compositions may also be in the form of oil-in-water emulsions. The oily phase may be a vegetable oil, for example olive oil or peanut oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate.

Dispersible Powders

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above.

Tablets and Capsules

Tablets typically comprise conventional pharmaceutically compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules (including time release and sustained release formulations) typically comprise one or more solid diluents disclosed above. The selection of carrier components often depends on secondary considerations like taste, cost, and shelf stability.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methylcellulose phthalate, ethyl cellulose, Eudragit coatings, waxes and shellac.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

### EXAMPLES

We describe two patients with borderline personality disorder and prominent cutting behaviors refractory to psychopharmacologic interventions. In both cases, the self-injurious behavior significantly decreased after initiating treatment with the glutamate modulating agent riluzole. In one case, treatment with NAC yielded a similar result in attenuating self-injurious thoughts/behaviors.

These case reports suggest the clinical efficacy of glutamate modulating agents in the treatment of self-injurious behavior and borderline personality disorder.

### Example 1

### Treatment of a 26-Year Old Female with Self-Injurious Behavior Using Riluzole and N-Acetyl Cysteine

Patient 1 was a 26-year-old woman with a history of post-traumatic stress disorder and borderline personality disorder. She had an approximately one and a half year history of self-injurious and cutting behaviors secondary to feelings of guilt, depression, anxiety and poor stress coping mechanisms. She was previously treated with intensive psychotherapy, cognitive behavioral therapy, and pharmacotherapy. Medication management included treatment trials, either alone or in combination, of the following medications: CELEXA (citraloprom HBr), PROZAC (fluoxetine HCl), REMERON (mirtazapine), ZYPREXA (olanzapine), KLONOPIN (clonazepam), LAMICTAL (lamotrigine), RISPERDAL (risperidone), Abilify (aripiprazole), EFFEXOR (venlafaxine HCl), XANAX (alprazolam), WELLBUTRIN (bubroprion HCl), and ATIVAN (lorazepam). Medications provided little relief and her cutting behaviors continued on a daily to weekly basis. Riluzole, a glutamate modulating agent, was added to her medication regimen which consisted of LEXAPRO (escitalopram) 20 mg a day and KLONOPIN 1 mg at bedtime. Riluzole was dosed at 50 mg twice a day. She reported a complete cessation of cravings to cut and was able to stop all self-injurious behaviors. This attenuation in her self-injurious behaviors occurred between week 2 and 3 of treatment with riluzole. She remained on the riluzole for a total of approximately eight weeks without a single episode or desire to harm herself.

While on riluzole Patient 1 experienced significant sedation. Despite the beneficial effects of riluzole with regard to the cutting/self-injurious behaviors (as well as cravings to cut), Patient 1 requested to stop the riluzole secondary to the sedation. Within 1 week of stopping the riluzole, her desire and cravings to cut returned. She was then treated with N-Acetyl Cysteine (NAC), another agent thought to modulate glutamatergic function. Within 2 weeks of treatment with NAC, her desire to cut remitted and she has not engaged in any self-injurious behaviors for over three months. Her self-report was that the NAC significantly attenuated the self-injurious cravings, but the desire to cut was not completely eradicated as they had been while on riluzole.

### Example 2

### Treatment of a 45-Year-Old Female with Self-Injurious Behavior Using Riluzole

Patient 2 was a 45-year-old woman with a long history of borderline personality disorder, major depression, obsessive-compulsive disorder and generalized anxiety disorder. She had a several year history of engaging in self-injurious behavior such as hitting herself until she bruised, banging her head, and cutting herself. These self-injurious behaviors and the cravings to cut worsened under times of stress. She was previously treated with intensive psychotherapy, cognitive behavioral therapy, and pharmacotherapy. Medication management included treatment trials, either alone or in combination, of the following medications: EFFEXOR, ZOLOFT (sertaline HCl), TOPAMAX (topirimate), ATIVAN (lorazepam), trazodone, ABILIFY AND NEURONTIN (gabapentin). Riluzole, a glutamate modulating agent, was added to her medication regimen which consisted of ZOLOFT 200 mg each day, RISPERDAL (risperidone) 2 mg at bedtime, KLONOPIN 1 mg three times per day and PROVIGIL (modafinil) 200 mg a day. Riluzole was dosed at 50 mg twice a day. She reported a marked attenuation and ultimately, complete cessation of the desire to engage in self-injurious behaviors. Similar to the first case, the cessation in self-injurious behaviors occurred between week 2 and 3 of treatment with riluzole. Patient 2 has not engaged in any self-injurious behaviors in over six months, which represents the longest period of time that she has not engaged in self-injurious behavior over the last several years.

What is claimed is:

1. A method of treating borderline personality disorder or self-injurious behavior in a patient in need thereof by administering an effective amount of a glutamate modulating agent.

2. The method of claim 1, wherein the self-injurious behavior is treated

3. The method of claim 1, wherein the glutamate modulating agent is riluzole or N-acetyl cysteine.

4. The method of claim 1 wherein the glutamate modulating agent is riluzole and from 10 mg to 300 mg riluzole are admininstered daily.

5. The method of claim 1, wherein the effective amount is an amount effective to decrease the number of episodes of self-injurious behavior or severity of self-injurious behavior in the patient.

6. The method of claim 1 wherein the glutamate modulating agent is administered together with one or more other CNS active agent.

7. The method of claim 6, wherein the other CNS active agent is an anti-psychotic, an anti-convulsant, a tricyclic antidepressant, a monoamine oxidase inhibitor, a selective serotonin reuptake inhibitor, a selective serotonin-norepinephrine reuptake inhibitor, a norepinephrine dopamine reuptake inhibitor, a serotonin-2 antagonist reuptake inhibitor, a benzodiazepine, a wakefulness promoting agent, or an anti-manic agent.

8. A method of using a glutamate modulating agent comprising informing a user that the glutamate modulating agent may be used to treat borderline personality disorder or self injurious behavior.

9. The method of claim 8 wherein the glutamate modulating agent is riluzole or N-acetyl cysteine.

10. The method of claim 8, additionally comprising providing the user with riluzole or N-acetyl cysteine.

11. The method of claim 8, additionally comprising providing riluzole or N-acetyl cysteine in a container and the informing is by reference to a package insert associated with the container.

12. The method of claim 8, wherein the informing is by reference to information material;
by reference to a package active agent insert, a flyer or an advertisement;
by presentation of information at a seminar, conference, or other educational presentation; or
by a conversation between a pharmaceutical sales representative and a medical care worker.

13. The method of claim 6, wherein the other CNS active agent is clozapine, olanzapine, risperidone, aripiprazole, amisulpride, loxazpine, clonazepam, gabapentin, lamotrigine, topiramate, doxepin, amitriptyline, amoxapine, clomipramine, desipramine, doxepin, imipramine, maprotiline, nortriptyline, protriptyline, trimipramine, isocarboxazid, phenelzine, tranylcypromine, citalopram, clovoxamine, duloxetine, escitalopram, femoxetine, flesinoxan, fluoxetine, fluvoxamine, paroxetine, setraline, trazodone, zimeldine, atomoxetine, mirtazapine, venlafaxine, nefazodone, bupropion, alprazolam, diazepam, lorazepam, flurazepam, flumazenil, bentazepam, carbamazepine, lithium, modafinil. aprepitant, or a combination of the foregoing.

14. A pharmaceutical composition comprising
(i) one of riluzole and N-acetyl cysteine; and
(ii) one or more other CNS active agent combined in a single dosage form.

15. The pharmaceutical composition of claim 14, wherein the one or more other CNS active agent is an anti-psychotic, an anti-convulsant, a tricyclic antidepressant, a monoamine oxidase inhibitor, a selective serotonin reuptake inhibitor, a selective serotonin-norepinephrine reuptake inhibitor, a norepinephrine dopamine reuptake inhibitor, a serotonin-2 antagonist reuptake inhibitor, a benzodiazepine, a wakefulness promoting agent, an anti-manic agent, or a combination of the foregoing.

16. The pharmaceutical composition of claim 14, wherein the one or more other CNS active agent is clozapine, olanzapine, risperidone, aripiprazole, amisulpride, loxazpine, clonazepam, gabapentin, lamotrigine, topiramate, doxepin, amitriptyline, amoxapine, clomipramine, desipramine, doxepin, imipramine, maprotiline, nortriptyline, protriptyline, trimipramine, isocarboxazid, phenelzine, tranylcypromine, citalopram, clovoxamine, duloxetine, escitalopram, femoxetine, flesinoxan, fluoxetine, fluvoxamine, paroxetine, setraline, trazodone, zimeldine, atomoxetine, mirtazapine, venlafaxine, nefazodone, bupropion, alprazolam, diazepam, lorazepam, flurazepam, flumazenil, bentazepam, carbamazepine, lithium, modafinil. aprepitant, or a combination of the foregoing.

17. The pharmaceutical composition of claim 14, wherein the single dosage form is a dosage form suitable for oral administration.

18. An article of manufacture comprising a glutamate modulating agent in a container and printed labeling stating that the glutamate modulating agent is useful for treating a borderline personality disorder or self-injurious behavior.

19. The article of manufacture of claim 18 wherein the glutamate modulating agent is riluzole or N-acetyl cysteine.

20. The article of manufacture of claim 17, additionally comprising one or more other CNS active agent.

21. The article of manufacture of claim 17, wherein the printed labeling is labeling approved by the United States FDA.
